(19) United States
(12) Patent Application Publication
(10) Pub. No.: US 2025/0044301 A1
(43) Pub. Date: Feb. 6, 2025

(54) DIAGNOSTIC METHOD FOR MULTIPLE SCLEROSIS

(71) Applicant: Deutsches Krebsforschungszentrum Stiftung des öffentlichen Rechts, Heidelberg (DE)

(72) Inventors: Harald ZUR HAUSEN, Heidelberg (DE); Timo BUND, Heidelberg (DE); Ethel-Michele DE VILLIERS-ZUR HAUSEN, Heidelberg (DE); Isolde SCHUSTER, Heidelberg (DE); Hans GRÖNLUND, Lidingo (SE)

(21) Appl. No.: 18/798,486

(22) Filed: Aug. 8, 2024

Related U.S. Application Data
(63) Continuation of application No. PCT/EP2023/053369, filed on Feb. 10, 2023.

(30) Foreign Application Priority Data
Feb. 10, 2022 (EP) 22156123.6

(51) Int. Cl. G01N 33/68; C12Q 1/28; G01N 33/543; G01N 33/58

(52) U.S. Cl. CPC G01N 33/6857; C12Q 1/28; G01N 33/54306; G01N 33/58; G01N 2333/46; G01N 2333/908; G01N 2800/285; G01N 2800/52

(57) ABSTRACT

The present invention provides a method for diagnosing and monitoring MS on the basis of an immunoassay using MSBI1.176 or MSBI2.176 Rep protein or fragments thereof as an antigen for binding antibodies against MSBI1.176 or MSBI2.176 Rep protein from a subject's sample.

### CROSS-REFERENCE TO RELATED APPLICATIONS

This application is a continuation of PCT/EP2023/053369, filed Feb. 10, 2023; which claims priority to European Patent Application No. 22156123.6, filed Feb. 10, 2022. The contents of the above-identified applications are incorporated herein by reference in their entireties.

### REFERENCE TO SEQUENCE LISTING, TABLE OR COMPUTER PROGRAM

This application contains an ST.26 compliant sequence listing, which is submitted concurrently in xml format via Patent Center and is hereby incorporated by reference it its entirety. The .xml copy created on Feb. 10, 2023, is named K3671PCT sequence listing.xml and is 14.6 kilobytes in size.

### TECHNICAL FIELD

The present invention concerns a diagnostic method for Multiple Sclerosis. The method is based on a bridging ELISA immunoassay for detecting antibodies against BMMF 1 Rep protein in a subject's sample.

### BACKGROUND OF THE INVENTION

Neurodegenerative diseases of the central nervous system (CNS) which cause progressive loss of neuronal structure and function are particularly devastating diseases for the affected patients and their families. Among these neurodegenerative diseases are, for example, Multiple Sclerosis (MS), Parkinson's disease, Alzheimer's disease, amyotrophic lateral sclerosis (ALS) and stroke. Due to the complexity of the CNS many of these diseases are only poorly understood to date.

One of the most common progressive neurodegenerative diseases is Multiple Sclerosis (MS). MS is a chronic inflammatory, demyelinating disease of the CNS and the leading cause of neurological disability in young or middle-aged adults. Multiple sclerosis (MS) is an autoimmune disease with the autoimmune activity directed against central nervous system (CNS) antigens. It affects approximately 2.3 million individuals worldwide (GBD 2016 Multiple Sclerosis Collaborators, Lancet Neurol. 2019, vol. 18, pp. 269-85) and currently no curative treatment is available. It is primarily a disease of young adults, with 70%-80% of patients having an age of initial clinical presentation to a physician between 20 and 40 years (Noonan et al., Neurology 2002, 58, pp. 136-138) although onset may occur earlier which, however, is often not recognized by the patient as an illness symptom. MS has a gender bias influenced by the phenotype, with approximately up to 64%-70% of diagnosed patients being women (Noonan et al. 2002).

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard textbooks of medicine and neurology.

The pathogenesis of MS has been attributed to a breakdown of T lymphocyte tolerance to CNS self-antigens resulting in chronic inflammation with subsequent demyelination and neuro-axonal degeneration predominantly in the periventricular and juxtacortical white matter (Compston, A. and Coles A. (2008) Lancet. 372:1502-1517). Axonal damage arises already early in the disease, also independent of demyelination and correlates best with clinical disability during the progressive course of MS (Kornek et al. (2000), Am J Pathol. 157:267-276). Similarly, neuronal damage and atrophy of the gray matter, with a predilection for the cingulate gyrus, the fronto-temporal cortices and the hippocampus, have also been shown to occur from the earliest stages and are likely to play an important role in clinical progression (Fisher et al. (2008), Ann Neurol. 64:255-265).

There are four major clinical types of MS: 1) relapsing-remitting MS (RR-MS), characterized by clearly defined relapses with full recovery or residual deficit upon recovery; periods between disease relapses characterized by a lack of disease progression; 2) secondary progressive MS (SP-MS), characterized by initial relapsing remitting course followed by progression with or without occasional relapses, minor remissions, and plateaus; 3) primary progressive MS (PP-MS), characterized by disease progression from onset with occasional plateaus and temporary minor improvements allowed; and 4) progressive relapsing MS (PR-MS), characterized by progressive disease onset, with clear acute relapses, with or without full recovery; periods between relapses characterized by continuing progression.

Clinically, the illness most often presents as a relapsing-remitting disease and, to a lesser extent, as steady progression of neurological disability. Relapsing-remitting MS (RR-MS) is characterized by recurrent attacks of focal or multifocal neurologic dysfunction. Attacks may occur, remit, and recur, seemingly randomly over many years. Remission is often incomplete and as one attack follows another, a stepwise downward progression ensues with increasing permanent neurological deficit. The usual course of RR-MS is characterized by repeated relapses associated, for the majority of patients, with the eventual onset of disease progression. The subsequent course of the disease is unpredictable, although most patients with a relapsing-remitting disease (RR-MS) will eventually develop secondary progressive disease. In the relapsing-remitting phase, relapses alternate with periods of clinical inactivity and may or may not be marked by sequelae depending on the presence of neurological deficits between episodes. Periods between relapses during the relapsing-remitting phase are clinically stable. On the other hand, patients with progressive MS exhibit a steady increase in deficits, as defined above and either from onset or after a period of episodes, but this designation does not preclude the further occurrence of new relapses.

The disease is characterized by inflammation in parts of the CNS, leading to the loss of the myelin sheathing around neuronal axons (demyelination), axonal loss, and the eventual death of neurons, oligodendrocytes and glial cells. For a comprehensive review of MS and current therapies, see, e.g., McAlpine's Multiple Sclerosis, by Alastair Compston et al., 4th edition, Churchill Livingstone Elsevier, 2006.

It was shown more recently that besides T cells, B lymphocytes (expressing CD20 molecule) may play a central role in MS and influence the underlying pathophysiology through at least four specific functions:
1. Antigen presentation: B cells can present self neuroantigens to T cells and activate them.
2. Cytokine production: B cells in patients with MS produce abnormal proinflammatory cytokines, which can activate T cells and other immune cells.
3. Autoantibody production: B cells produce autoantibodies that may cause tissue damage and activate macrophages and natural killer (NK) cells.
4. Follicle-like aggregate formation: B cells are present in ectopic lymphoid follicle-like aggregates, linked to microglia activation, local inflammation, and neuronal loss in the nearby cortex.

Despite the number of studies on the disease and a multidisciplinary approach to the problem, some pathogenic mechanisms of multiple sclerosis are still obscure and the etiology is unknown.

There is a recent article of K. Bjornevik et al. (Science 10, 1126/science.abj8222 (2022)) reporting about an analysis of 10 million blood samples of young adults on active duty in the US military. 955 of whom were diagnosed with MS during their period of duty. This article mentions that the risk of MS increased 32-fold after infection with Epstein Barr Virus (EBV) but was not increased after infection with other viruses, including the similarly transmitted cytomegalovirus (CMV). In an accompanying commentary, epidemiologist Alberto Ascherio, senior author of the study concludes that the bottom-line is almost “if you are not infected with EBV, you don't get MS. It's rare to get such black-and-white results.” However, others wrote in their commentary to this article that even if EBV is the triggering event for MS, infection alone is insufficient for a diagnosis because of the ubiquitous nature of EBV where about 95% of adults have undergone an EBV infection (zur Hausen et al., 2017, Curr. Top. Microbiol. Immunol., Volume 407, 83-116; Borkosky et al., 2012, PLOS One 7 (2): e32160, 2012), mostly during childhood or as young adults—a circumstance which was also discussed by the authors of the Bjornevik et al. article themselves.

There are several treatments approved by regulatory agencies for relapsing-remitting multiple sclerosis which shall modify the course of the disease. These treatments include interferon beta, glatiramer acetate, mitoxantrone, natalizumab, fingolimod, teriflunomide, dimethyl fumarate, alemtuzumab, rituximab and daclizumab. The interferons and glatiramer acetate are first-line treatments that reduce relapses by approximately 30% (see, e.g., Tsang 2011, The Australian family physician 40 (12): 948-55). Natalizumab reduces the relapse rate more than the interferons, however, due to issues of adverse effects it is a second-line agent reserved for those who do not respond to other treatments or patients with severe disease (see, e.g., Tsang 2011, loc. cit.). Treatment of clinically isolated syndrome (CIS) with interferons decreases the chance of progressing to clinically definite MS (Compston 2008, Lancet 372 (9648): 1502-17). Recently, new monoclonal antibodies such as ocrelizumab, alemtuzumab and daclizumab have shown potential as therapeutics for MS. The anti-CD20 B-cell targeting monoclonal antibody ocrelizumab has shown beneficial effects in both relapsing and primary progressive forms of MS in phase II and III clinical trials (NCT00676715, NCT01247324, NCT01412333, NCT01194570)

MS diagnosis is based at present on clinical investigations by a medical practitioner. Such investigations involve testing of the capabilities of a patient for certain physical activities. Several tests have been developed and are routinely applied by medical practitioners. These tests aim at assessing walking, balance, and other motoric abilities. Examples of currently applied tests are the Expanded Disability Status Scale (EDSS) or Multiple Sclerosis Functional Composite (MSFC). These tests require the presence of a medical practitioner for evaluation and assessment purposes and are currently performed ambulant at doctor's offices or hospitals. Recently, there have been some efforts in monitoring MS patients using smartphone devices in order to collect data of MS patients in a natural setting (Bove 2015, Neurol Neuroimmunol Neuroinflamm 2 (6): 62). Further, diagnostic tools are used in MS diagnosis. Such tools include neuroimaging, analysis of cerebrospinal fluid and evoked potentials. Magnetic resonance imaging (MRI) of the brain and spinal cord can visualize demyelination (lesions or plaques). Contrast agents containing gadolinium can be administered intravenously to mark active plaques and, differentiate acute inflammation from the existence of older lesions which are not associated with symptoms at the moment of the evaluation. The analysis of cerebrospinal fluid obtained from a lumbar puncture can provide evidence of chronic inflammation of the central nervous system. The cerebrospinal fluid can be analyzed for oligoclonal immunoglobulin bands, which are an inflammation marker present in 75-85% of people with MS (Link 2006, J Neuroimmunol. 180 (1-2): 17-28). However, none of the aforementioned techniques is specific to MS. Therefore, ascertainment of diagnosis may require repetition of clinical and MRI investigations to demonstrate dissemination in space and in time of the disease which is a prerequisite to MS diagnosis. In addition, none of the above mentioned methods is suited for the diagnosis of early phases of MS when the patient has already some (unspecific) symptoms but still no MRI-visible demyelination signs in the brain. The current MS diagnostic procedure is rather long and tortuous. Thus, there is the need for an early diagnosis of MS since an earliest possible therapeutic intervention would be most effective for the long term, even with currently available therapies. In addition, there is presently no easy and cost-sensitive method for monitoring the disease progress or therapeutic success.

Thus, the technical problem underlying the present invention may be seen in the provision of means and methods complying with the aforementioned needs.

The technical problem is solved by the embodiments characterized in the claims and described herein below.

Although Bjornevik et al., 2022 conclude that their experiments demonstrate that EBV is the cause of MS, they do not exclude the contribution of other factors.

The presence of an additional factor was already considered by the inventors in an earlier patent application and in the literature (WO 2016/005054 A2 (=US 2020/0002723 A1); zur Hausen et al., Nature Reviews Clinical Oncology, 2015, zur Hausen et al., Curr. Top. Microbiol. Immunol. 2017, Volume 407, 83-116). Thus, according to the inventors MS results from
Latent infections of the same cell with two different infectious agents, one of them most likely EBV, the other one acquired by bovine milk and meat consumption (bovine meat and milk factor—BMMF) as the first event. Each of them latently infects individual cells, but occasionally genomes of both agents occur within the same cell;
Reactivation of the EBV to a lytic cycle as a second precondition. For EBV this is probably linked to increased levels of transforming growth factor β (TGF β) which is negatively regulated by activated vitamin D receptors;
As third event, amplification of BMMF, resulting in partial suppression of EBV DNA synthesis and formation of BMMF proteins or particles or spreading of its nucleic acid to neighbouring cells via neuronal interconnections;
This is followed by an infection of neighbouring cells with expression of BMMF protein;
Finally, T-cell response against BMMF leads to the destruction of affected cells and in case of MS to plaque formation. This supports the clinical observation of the focal appearance of lesions, commonly starting from a central vein and the intensive localized immune response in early lesions.

A spectrum of 19 different, but partially related DNA molecules of BMMF subgroup 1 were isolated from different test material (multiple sclerosis (MS) brain tissue, bovine sera, milk) (Funk et al., 2014, Genome Announc 2 (4): e00846-14; Gunst et al. 2014, Genome Announc 2 (4): e00847-14; Lamberto et al. 2014, Genome Announc 2 (4): e00848-14; Whitley et al. 2014, Genome Announc 2 (4); e00849-14; Falida et al. 2017, Genome Announc 5 (17): e00266-17, zur Hausen, H., et al. 2017, Curr. Top. Microbiol. Immunol., Volume 407, 83-116).

Among these isolates, two DNA molecules were isolated from the brain tissue of one MS patient (Whitley et al. 2014, Genome Announc 2 (4); e00849-14). These isolates were called MSBI1.176 (MSBI, multiple sclerosis brain isolate) (1,766 bp) and MSBI2.176 (1,766 bp) and designated as “MSBI1 genome” and “MSBI2 genome”, respectively. Both belong to the BMMF family. The large open reading frames (ORFs) of the isolates encode a putative DNA replication protein sharing high similarity between them (de Villiers et al. Emerg. Microbes Infect. 2019, 8:1205-1218). Another common feature is the presence of iteron-like tandem repeats. The alignment of this repeat region indicates a variation in the core of single nucleotides (zur Hausen et al., Curr. Top. Microbiol. Immunol. 2017, Volume 407, 83-116). These iteron-like repeats may constitute the binding sites for Rep proteins. The sequences of the isolates have been deposited in the EMBL Databank under accession numbers LK931491 (MSBI1.176) and LK931492 (MSBI2.176) (Whitley C. et al. 2014) and have been aligned and described in WO 2016/005054 A2 (=US 2020/0002723 A1).

The isolation of 2 BMMF types from an autopsy lesion of MS (Whitley et al. 2014, Genome Announc 2 (4); e00849-14) seem to point to their possible involvement in MS etiology. It is a property of Herpes type viruses to amplify co-infecting small single to double-stranded DNAs of other DNA-containing agents (Schlehofer at al., 1986, Heilbronn et al., 1993). More recently, DNA amplification of small single-stranded TT virus by EBV has been reported (Borkosky et al. 2012). Thus far, TT-viruses have not been linked to MS. Yet, the genomic structural similarity of BMMFs, in addition to finding the latter in MS lesions and elevated BMMF antibody titers of MS patients in comparison to non-MS controls resulted in their inclusion of BMMF amplification, induced by reactivation of EBV as a second factor. Therefore, EBV-induced amplification was included into the above-mentioned model of MS pathogenesis (zur Hausen et al. 2017).

The present inventors have found out that the MSBI1 or MSBI2 genome shows a significant production of transcribed RNA and that MSBI1 or MSBI2 genome-encoded Rep protein is expressed in human cells (Eilebrecht et al., Scientific Reports 8:2851, 2018). Additionally, serology on human samples identified antibodies reactive against the MSBI1 protein. As already mentioned above, according to the inventors' hypothesis, MS results from latent infections of the same cell with two different agents, one of them EBV and the other one acquired by bovine milk consumption (zur Hausen et al., Nature Reviews Clinical Oncology, 2015, zur Hausen et al., Curr. Top. Microbiol. Immunol. 2017, Volume 407, 83-116). The latter one is called bovine milk and meat factor (BMMF). Finally, T-cell response against BMMF, in particular against MSBI1 Rep, leads to the destruction of affected cells and in case of MS to plaque formation (zur Hausen et al., Nature Reviews Clinical Oncology, 2015, zur Hausen et al., Curr. Top. Microbiol. Immunol. 2017, Volume 407, 83-116). This supports the clinical observation of the focal appearance of lesions, commonly starting from a central vein and the intensive localized immune response in (early) lesions (Moreno M A et al., Neurol Neuroimmunol Neuroinflamm. 2018; 5 (4): e466). Thus, the detection of this immune response should be a suitable means for diagnosing and monitoring the progress of MS.

Thus, the present invention provides a method for diagnosing and monitoring MS on the basis of an immunoassay using MSBI1.176 or MSBI2.176 Rep protein or fragments thereof as an antigen for binding antibodies against MSBI1.176 or MSBI2.176 Rep protein from a subject's sample.

The invention provides a method for the immunological determination of MS in a sample using a bridging immunoassay comprising a capture antigen and a tracer antigen (also called detection antigen).

In a preferred embodiment of the invention, the capture antigen is immobilized via a specific binding pair to a solid phase. Such a binding pair (first component/second component) is, for example, Streptavidin or Avidin/Biotin, antibody/antigen (see, for example, Hermanson, G. T., et al., Bioconjugate Techniques, Academic Press, 1996), lectin/polysaccharide, steroid/steroid binding protein, hormone/hormone receptor, enzyme/substrate, IgG/Protein A and/or G, etc. Preferably, the capture antigen is conjugated to biotin and immobilization is performed via immobilized avidin or streptavidin, most preferably streptavidin.

In a preferred embodiment the tracer antigen may be replaced by an anti-human antibody, preferably an anti-human IgG or IgM antibody. These anti-human IgG or IgM antibodies are mostly raised in goat, mouse or rabbit and may be monoclonal or polyclonal. Numerous examples of suitable anti-human IgG or IGM antibodies are commercially available, e.g. from Thermo Fisher, Promega, Abcam or Jackson Immuno Research.

Preferably, the tracer antigen/antibody is labelled with a detectable label or detectable tag.

Examples for detectable labels/tags are chromogens (fluorescent or luminescent groups and dyes), enzymes, NMR-active groups or metal particles. The detectable label/tag can also be a photoactivatable crosslinking group, e.g. an azido or an azirine group. Particularly preferred is the labelling with horseradish peroxidase (HRP).

Both the binding of an antigen to a solid phase via Biotin/Streptavidin and the HRP-labelling of an antigen are standard procedures and well known to the person skilled in the art. In brief, coupling of biotin (N-Hydroxysuccinimide Ester (NHS)-biotin) is performed via primary amines of lysine residues at pH 4-10 with an excess of at least 20× when compared to the antigen (see e.g. documentation on Thermo Scientific™ EZ-Link™ NHS-Biotin biotinylation or Diner, I. et al., J Biol Chem. 2014-12-19; 289 (51): 35296-35313). Alternatively, at acidic pH, coupling of carboxyl groups with EDC and Thermo Scientific™ EZ-Link™ Hydrazid-Biotin is performed (based on the documentation by Thermo Scientific™ EZ-Link™ Hydrazid-Biotin used in Roncato F et al. Nat Commun 2018, 9:4070). HRP-coupling is performed based on a 2-step process, first adding N-succinimidyl S-acetylthioacetate (SATA) to the target antigens at primary amines of e.g. lysins to introduce protected sulfhydryl groups at pH 4-10 (see e.g. documentation on Thermo Scientific™ Pierce™ SATA (N-succinimidyl S-acetylthioacetate) used in Duncan, R. J. S., et al. Anal Biochem (1983) 132:68-73). In a second step maleimide-activated HRP is coupled to the antigen via the previously introduced sulfhydryl groups (see e.g. documentation on Thermo Scientific™ EZ-Link™ Maleimide Activated Horseradish Peroxidase conjugation, Pleiner, T., Bates, M., Görlich, D, J Cell Biol 2018; (217): 3 1143-1154). In an alternative technical setup, preferably at basic pH, Thermo Scientific™ EZ-Link™ Plus Activated Peroxidase is used in a single step process to couple HRP to primary amines (see e.g. documentation on Thermo Scientific™ EZ-Link™ Plus Activated Peroxidase coupling e.g. in Chen H et al. Journal: J Immunol Res 2018, 2018:4894705).

In an alternative embodiment, to increase stability of the Biotin Rep antigen over longer periods of time (months to years, long-term storage) and to simplify and shorten the work (SOP) for the end-user, the technical option to immobilize, stabilize and store the biotin Rep oligomers on the microtiter plates was implemented. In such a way, the immobilization of the Biotin Rep to the streptavidin microtiter plate is already prepared and not to be covered by the user. Therefore, specific sugar formulations (containing e.g. 10-30% (e.g. 20%) saccharose, 0.01-0.1% (e.g. 0.06%) 5-Bromo-5-nitro-1,3-dioxane, and optionally 1% (porcine) gelatine) were tested to allow immobilization of Biotin Rep oligomers on the microtiter plates with subsequent drying of the material and storage as well as confirmation of adequate detection of BMMF antibodies after reactivation of the material when used in the diagnostic test setup. In this regard reference is made to Example 4.

The term “sample” includes, but is not limited to, whole blood, serum, or plasma from an individual suspected of having MS or having already a confirmed diagnosis of MS. The sample can for example be a “post-treatment” sample wherein the sample is obtained after one or more treatments, or a “base-line sample” which is for example used for a first diagnosis or as a base line for assessing disease progression.

The term “subject” or “patient” as used herein relates to a human individual that shall suffer from or shall be suspected to suffer from MS, i.e. it may already show some or all of the symptoms generally associated with the said disease.

The term “solid phase” means a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; cuvettes, tubes, or other spectrometer sample containers. A solid phase component of an assay is distinguished from inert solid surfaces with which the assay may be in contact in that a “solid phase” contains at least one moiety on its surface, which is intended to interact with the capture antigen. A solid phase may be a stationary component, such as a tube, strip, cuvette, or microtiter plate, or may be a non-stationary component, such as beads and microparticles. According to the present invention the solid phase is most preferably a microtiter plate.

Immunoassays are well known to the skilled artisan. Methods for carrying out such assays as well as practical applications and procedures are summarized in related textbooks (e.g. Colowick, S. P. and Caplan, N. O. (eds.), “Methods in Enzymology”, Academic Press, dealing with immunological detection methods). In an embodiment, the immunoassay is an enzyme linked immunosorbent assay (ELISA). In a preferred embodiment the immunoassay is a bridging ELISA. It may be considered that it works similar to an “Anti-Drug Antibody Assay” (ADA Test).

The term “bridging ELISA” as used herein refers to an ELISA comprising a solid support and a capture antigen or fragment thereof (specific for the antibodies in the subject's sample) immobilized onto the solid support. In such an ELISA an amount of target antibody (e.g. anti-MSBI1 or 2 Rep antibody comprised in a sample) is bound by the capture antigen. The bound antibody forms through binding to a second labelled antigen or fragment thereof (i.e. a tracer antigen or fragment thereof) an antigen-antibody-antigen complex that is detected through a substrate reaction, e.g. dye reaction generated based on the labelled antigen, which can alternatively constitute a chemiluminescence or fluorescence label for detection of the antigen-antibody-antigen complex.

The term “epitope” as used herein refers to the site on the antigen that is recognized by the antibodies or binding fragments.

The term “control” as used herein refers to a sample from a subject or a group of subjects who are not having the disease or having any symptoms. The terms “healthy control” or “control” are used interchangeably.

The term “non-denaturated” as used herein refers to a protein in its native conformation (e.g. 3D conformation) or in a conformation sufficient to confer functionality.

The term “reference agent” or “calibration agent” as used herein refers to an agent that can be used in an assay for calibration purposes. It can be for example a known antibody against MSBI 1 Rep. Examples are Rep-specific mouse-monoclonal antibodies AB 8 or AB11 (Bund et al., PNAS 2021 Vol. 118 No. 12 e2025830118) or those mentioned in WO 2018/069296 A2 (=US 2019/0270795 A1), in particular AB 3-6 (deposited as “MSBI1 381-6-2” with the DSMZ Braunschweig under the accession number DSM ACC 3329). These reference agents are also helpful as quality controls for the purity of the antigen preparations. For example, AB 3-6 shows a significantly increased reactivity for oligomeric Rep antigens in the bridging ELISA; AB 11 shows a high reactivity with aggregate antigens in the bridging ELISA and AB 8 reacts both with oligomers and aggregates.

The term “vector” encompasses any vehicle suitable for recombinantly expressing a protein and/or fragment thereof., Possible expression vectors include but are not limited to cosmids, plasmids, or modified viruses (e.g. replication defective retroviruses, adenoviruses and adeno-associated viruses).

The term “host cell” includes a wide variety of prokaryotic and eukaryotic host cells. For example, the proteins of the invention may be expressed in bacterial cells such as E. coli, insect cells (using baculovirus), yeast cells or mammalian cells.

“Rep protein” as used herein refers to a DNA-replication-associated protein (RepB). The Rep protein comprises DNA binding activity and could be essential for initiation of replication of episomal/viral DNA molecules. In general Rep protein refers to a Rep protein from the group of the Small Sphinx Genome (Whitley et al., 2014). In particular, the Rep protein is a wild-type MSBI1 or MSBI2 Rep protein, or a mutant MSBI1 or MSBI2 Rep protein having two point mutations (MSBI1 Rep 27/154E or MSBI2 Rep 27/155E) compared to the wild-type sequences. Preferably, the wild-type MSBI1 Rep protein has the amino acid sequence as depicted in SEQ ID NO:1 and is derived from MSBI1.176 deposited in the EMBL databank under the acc. no. LK931491. Preferably, the wild-type MSBI2 Rep protein has the amino acid sequence as depicted in SEQ ID NO: 4 and is derived from MSBI2.176 deposited in the EMBL databank under the acc. no. LK931492. Preferably, the mutant MSBI1 Rep protein has the amino acid sequence as depicted in SEQ ID NO: 3 and the MSBI2 Rep protein has the amino acid sequence as depicted in SEQ ID. No. 5. “Rep protein” also encompasses fragments and variants of the protein which are capable of binding an anti-Rep antibody specific for Rep protein having the amino acid sequence of SEQ ID NO: 1, 3, 4 or 5. Preferably, such a fragment is an immunogenic fragment of the protein having the amino acid sequence of SEQ ID NO: 1, 3, 4 or 5 which encompasses at least one epitope for an anti-Rep protein antibody against the Rep protein of SEQ ID NO:1, 3, 4 or 5 and, preferably, comprises at least 7, 8, 9, 10, 15, 20, 25 or 50 contiguous amino acids. In particular embodiments the fragment comprises or consists essentially of a domain of the Rep protein, for example, the N-terminal conserved region, the C-terminal variable region, the first or second DNA binding domain. A variant of the protein comprises one or more amino acid deletions, substitutions or additions compared to SEQ ID NO:1, 3, 4 or 5 and has a homology of at least 90%, 91%, 92%, 93%, 94%, 95%, 96%, 97%, 98% or 99% to the amino acid sequence of SEQ ID NO:1, 3, 4 or 5, wherein the variant is capable of binding an anti-Rep antibody specific for a Rep protein having the amino acid sequence of SEQ ID NO:1, 3, 4 or 5. Included within the definition of variant are, for example, polypeptides containing one or more analogues of an amino acid (including, for example, unnatural amino acids, peptide nucleic acid (PNA), etc.), polypeptides with substituted linkages, as well as other modifications known in the art, both naturally occurring and non-naturally occurring. The term “Rep protein” includes fusion proteins with a heterologous amino acid sequence, with a leader sequence or with a Tag-sequence and the like.

The synthesized wild-type or mutant MSBI1 or MSBI2 Rep proteins, including the Rep fragments and Rep variants as defined above, can be prepared by classical chemical synthesis. The synthesis can be carried out in homogeneous solution or in solid phase. The polypeptides according to this invention can also be prepared by means of recombinant DNA techniques. For example, the mutant protein (MSBI1 Rep 27/154E or MSBI2 Rep 27/155E) was engineered in which the aggregation potential of two amino acid sequences between residues 25-31 for MSBI1 & 2 Rep (RLILLAII) and residues 151-155 for MSBI1 Rep and residues 152-156 for MSBI2 Rep (LLICW) was minimized by two single point mutations. The basis for the cloning was a MSBI1 or MSBI2 Rep DNA sequence which was codon-optimized for Rep expression in the human system encoding the original wild-type MSBI1 or MSBI2 Rep primary amino acid sequence. The nucleotides coding for amino acid 27 (L, Leucine, DNA codon CTA) as well as the nucleotides coding for amino acid 154 (C, Cysteine, DNA codon TGT) were substituted by nucleotides coding for the amino acid glutamic acid (E, DNA codon GAG) in MSBI1.176. The nucleotides coding for amino acid 27 (L, Leucine, DNA codon CTA) as well as the nucleotides coding for amino acid 155 (C, Cysteine, DNA codon TGT) were substituted by nucleotides coding for the amino acid glutamic acid (E, DNA codon GAG) in MSBI2.176. For further details reference is made to WO 2019/008052 A2.

If not stated otherwise in the present application, “MSBI Rep” means both MSBI 1 Rep and MSBI 2 Rep protein, in particular from MSBI 1.176 and MSBI 2.176.

As mentioned above, the expression of both the wild-type and mutant Rep proteins may be made by recombinant DNA techniques. These are well known to a person skilled in the art. Suitable vectors and host cells are well known and commercially available. In addition, reference is made to Example 1 where the expression and purification of the proteins are described. In a particular preferred embodiment, the wild-type and mutant Rep proteins are separately expressed in a host cell, preferably E. coli, wherein the respective nucleic acids are codon-optimized for expression in E. coli (SEQ ID. Nos. 7 and 8). The recombinant production provides a mixture of Rep oligomers (1-4 Rep subunits) and aggregates (5-9 Rep subunits) for both the wild-type and mutant Rep proteins. However, due to the two point mutations in the mutant sequence it tends to form less aggregates than the wild-type. The non-denaturing purification of the mutant Rep oligomer and aggregate mixture is performed at pH 5.0-9.8, most preferably between pH 7.0-9.5, preferably in a buffer containing 30 ml 1×PBS pH 7.4, 300 mM NaCl, 1% Triton X-100, 5 mM imidazole, 5 mM β-mercaptoethanol, containing benzonase and proteinase inhibitors and loading on an affinity chromatography column. The elution and storage of the mutant Rep oligomer and aggregate mixture is made in a pH range of pH 4-11, preferably pH 4.8-9.8, most preferably between pH 5.0-9.5. The non-denaturing purification of the wild-type Rep oligomer and aggregate mixture is performed at pH 5.0-9.8, most preferably between pH 7.0-9.5, preferably in a buffer containing 30 ml 1×PBS pH 7.4, 300 mM NaCl, 1% Triton X-100, 5 mM imidazole, 5 mM β-mercaptoethanol, containing benzonase and proteinase inhibitors and loading on an affinity chromatography column. The elution and storage of the wild-type Rep oligomer and aggregate mixture is made in a pH range of pH 4-11, preferably pH 4.8-9.8, most preferably between pH 5.0-9.5 or pH 4.0-6.0. The whole preparation from the mutant and one half of the preparation from the wild type are separately subjected to biotinylation according to standard procedures as mentioned above. The other half of the wild-type preparation is coupled to HRP according to standard procedures as mentioned above. The biotinylated mutant preparation and the biotinylated wild-type preparation are separately subjected to size exclusion chromatographies (SEC) to separate the biotinylated oligomers and aggregates from each other. This leads to mutant biotinylated oligomers (MBO), mutant biotinylated aggregates (MBA), wild-type biotinylated oligomers (WBO) and wild-type biotinylated aggregates (WBA). In addition, the HRP-coupled wild-type preparation is subjected to size exclusion chromatography to separate the HRP-coupled oligomers (HRP-O) and aggregates (HRP-A) from each other. These different molecules are used to establish two immunoassays:
One is an oligomeric assay where the mutant biotinylated oligomers (MBO) are bound to a 96-well plate coated with streptavidin. Then the subject's sample is added to the plate concurrently or in quick succession with the HRP-coupled oligomers (HRP-O). After incubation and washing the substrate reaction is started for the detection of oligomer-reactive antibodies to quantify the antibody levels against MSBI1 Rep or MSBI2 Rep within the serum.
The other is the aggregate assay where the wild-type biotinylated aggregates (WBA) are bound to a 96-well plate coated with streptavidin. Then the subject's sample is added to the plate concurrently or in quick succession with the HRP-coupled aggregates (HRP-A). After incubation and washing the substrate reaction is started for detection of aggregate-reactive antibodies to quantify the antibody levels against MSBI1 Rep or MSBI2 Rep within the serum.

The assays may be calibrated with known antibodies against MSBI 1 Rep. Examples are Rep-specific mouse-monoclonal antibodies AB 8 or AB 11 (Bund et al., PNAS 2021 Vol. 118 No. 12 e2025830118) or those mentioned in WO 2018/069296 A2 (=US 2019/0270795 A1) or WO 2019/008052 A1 (=US 2020/0123520 A1), in particular AB 3 (deposited as “MSBI1 381-6-2” with the DSMZ Braunschweig under the accession number DSM ACC 3329).

Sera containing increased amounts of anti-Rep antibodies indicate that the corresponding subject was definitely exposed to Rep-related proteins or himself expressed Rep during a time period long enough to initiate a Rep specific immune response.

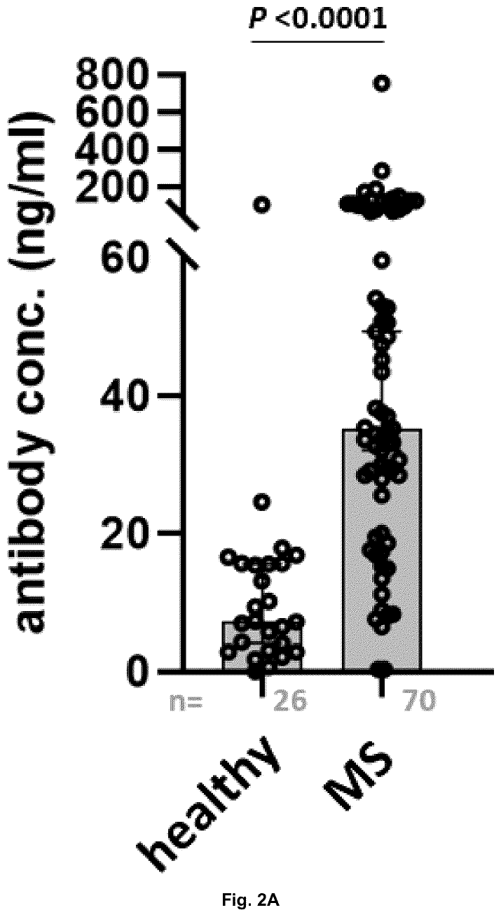

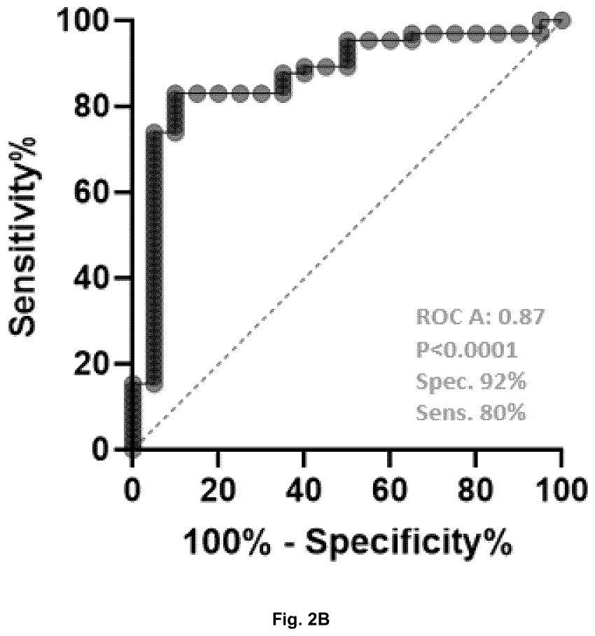

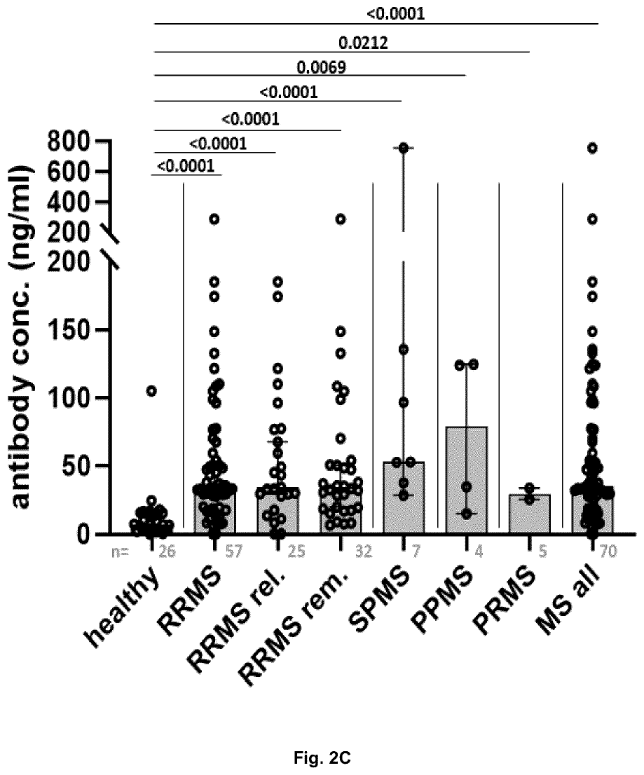

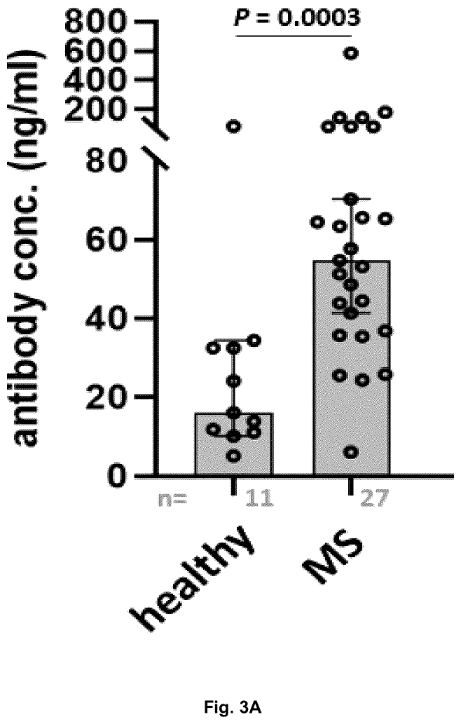

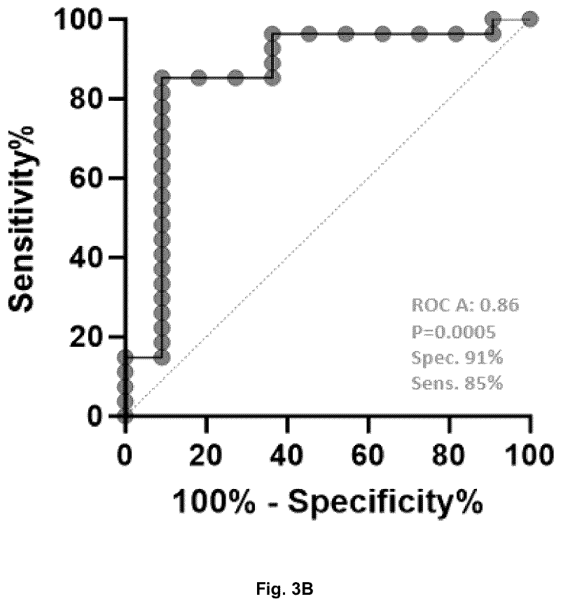

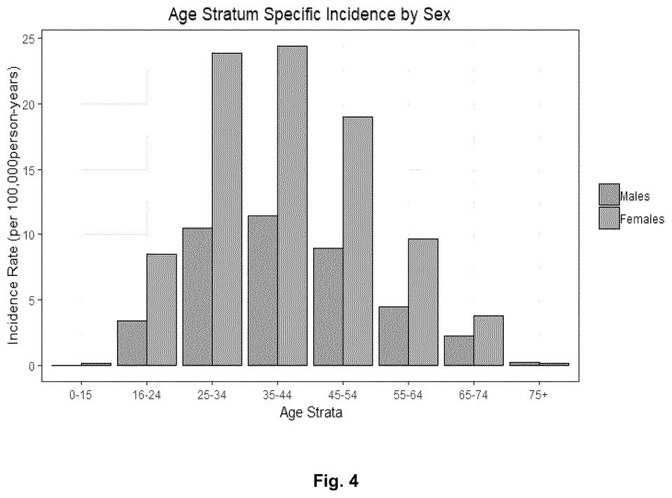

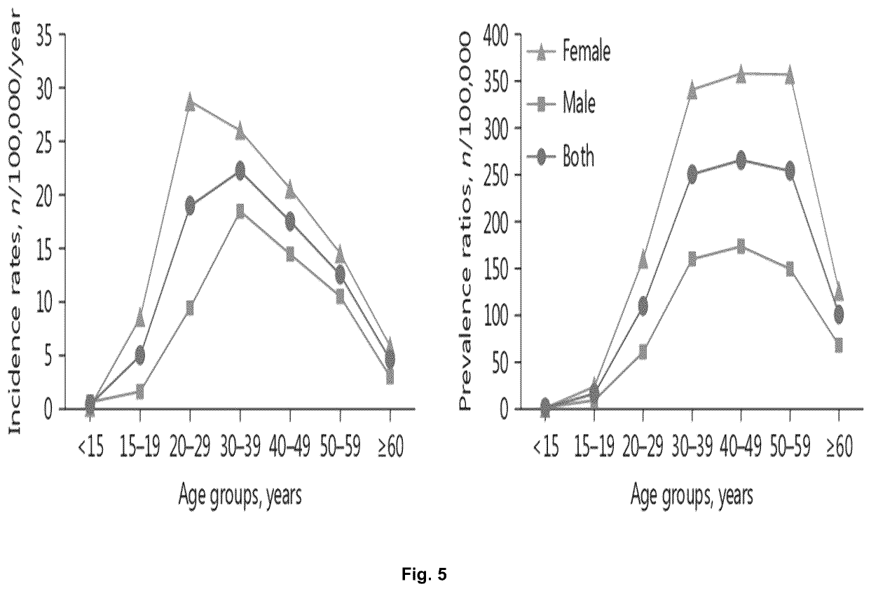

In the course of the present invention the assays have been validated with a number of blood samples from healthy individuals (=control) and patients having a confirmed MS history. The results are shown in FIGS. 2 and 3. In FIG. 2 the age of the patients and controls has been restricted to younger or equal to 53 years since it is generally known (c.f. FIGS. 4 and 5) that after the age of 55 the incidence of MS is rapidly decreasing. In addition, in FIG. 3 a restriction to females under or equal to the age 40 years has been applied since MS becomes more and more a disease of young females (c.f. FIGS. 4 and 5). Thus, it may be concluded that the aggregate assay is particularly suitable for young females whereas the oligomeric assay appears to be useful for both males and females until the age of about 55 years.

The assays of the present invention are suitable for diagnosing or monitoring all possible active or inactive forms of MS, i.e. relapsing-remitting MS (RR-MS), secondary progressive MS (SP-MS), 3) primary progressive MS (PP-MS) and/or progressive relapsing MS (PR-MS). Reference is made to FIG. 2 (c).

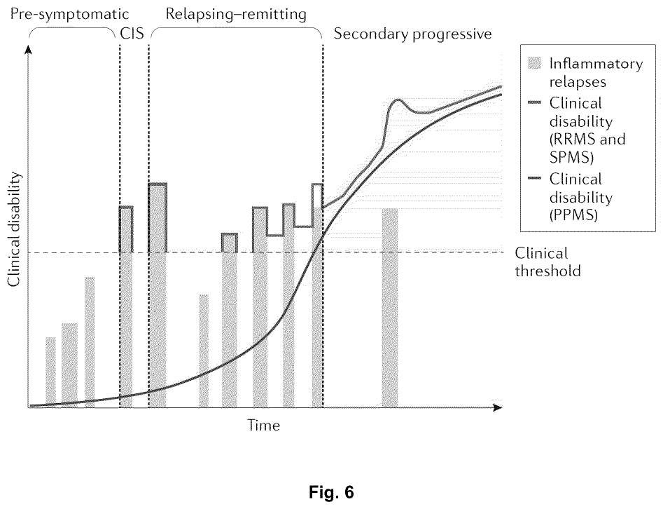

In addition, the assays are suited for a first diagnosis of MS, in particular during the pre-symptomatic phase (early diagnosis), and for monitoring the disease progression and/or treatment success, i.e. for therapy monitoring. The suitability of the assays of the present invention for early diagnosis is important since many patients show clinically isolated syndromes (CIS) which, however, do not fulfill the common diagnostic criteria and, thus, will not be considered as MS (c.f. FIG. 6). These pre-symptomatic patients loose important time until a treatment will be initiated due to a later diagnosis with a possible progress of the symptoms. In this regard reference is also made to the article of Compston et al. (2008, Lancet 372 (9648): 1502-17) where it has been confirmed that the treatment of clinically isolated syndrome (CIS) with interferons decreases the chance of progressing to clinically definite MS.

In addition, the assays are suitable for monitoring the disease progress and during therapy the therapeutic success.

The present invention also provides an ELISA kit that comprises (a) MSBI 1 Rep or MSBI 2 Rep protein or fragment thereof as a capture antigen that is immobilized via a specific binding pair to a solid phase, (b) MSBI 1 Rep or MSBI 2 Rep protein or fragment thereof that is conjugated to a detectable label as a tracer antigen, (c) a dye, chemiluminescent or fluorescent substrate and (d) instructions for using the kit in a method for diagnosing or monitoring Multiple Sclerosis.

In the present disclosure the term “a” should be understood to mean “at least one”. Terms of degree, such as “substantially”, “about” and “approximately”, as used herein mean a reasonable amount of deviation of the modified term such that the end result is not significantly changed. These terms of degree should be construed as including a deviation of at least ±5% of the modified term if this deviation would not negate the meaning of the word it modifies.

As used herein, the terms “include,” “includes,” and “including,” are meant to be non-limiting and are understood to mean “comprise,” “comprises,” and “comprising,” respectively.

The consecutive labeling of method steps as provided herein with numbers and/or letters is not meant to limit the method or any embodiments thereof to the particular indicated order. Various publications, including patents, patent applications, published patent applications, accession numbers, technical articles and scholarly articles are cited throughout the specification. Each of these cited references is herein incorporated by reference, in its entirety and for all purposes, herein.

The following figures are illustrative of the present disclosure:

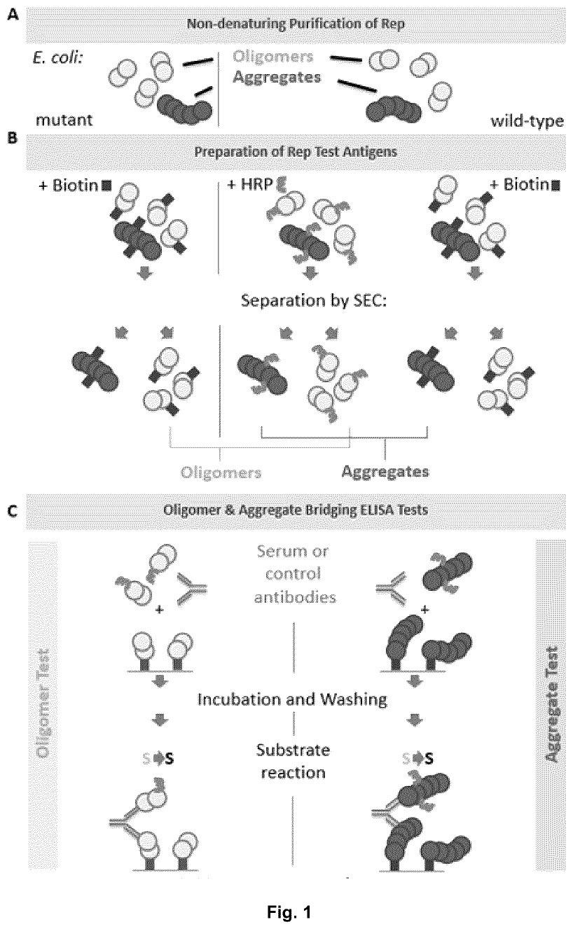

FIG. 1: Overview on the production of BMMF Rep antigens and the principle of BMMF-Rep specific serum tests based on a bridging ELISA assay setup

Rep oligomers (1-4 Rep subunits) and aggregates (5-9 Rep subunits) are produced by non-denaturing purification and subsequent buffering to pH 4-11 for the mutant. Rep oligomers (1-4 Rep subunits) and aggregates (5-9 Rep subunits) are produced by non-denaturing purification and subsequent buffering to pH 4-11 for the wild-type. The whole preparation from the mutant and one half of the preparation from the wild-type are separately subjected to biotinylation. The other half of the wild-type preparation is coupled to HRP. The biotinylated mutant preparation and the biotinylated wild-type preparation are separately subjected to size exclusion chromatographies (SEC) to separate the biotinylated oligomers and aggregates from each other. This leads to mutant-biotinylated oligomers (MBO), mutant-biotinylated aggregates (MBA), wild-type biotinylated oligomers (WBO) and wild-type biotinylated aggregates (WBA). In addition, the HRP-coupled wild-type preparation is subjected to size exclusion chromatography to separate the HRP-coupled oligomers (HRP-O) and aggregates (HRP-A) from each other.

Oligomeric test: The mutant-biotinylated oligomers (MBO) are bound to a 96-well plate coated with streptavidin. Then the subject's sample is added to the plate concurrently or in quick succession with the HRP-coupled oligomers (HRP-O). After incubation and washing the substrate reaction is started for detection of oligomer-reactive antibodies to quantify the antibody levels within the serum. The calibration of the test is achieved with a test sample containing a known antibody, i.e. Rep-specific mouse-monoclonal antibody AB 8 (Bund et al., PNAS 2021 Vol. 118 No. 12 e2025830118).

Aggregate test: The wild-type biotinylated aggregates (WBA) are bound to a 96-well plate coated with streptavidin. Then the subject's sample is added to the plate concurrently or in quick succession with the HRP-coupled aggregates (HRP-A). After incubation and washing the substrate reaction is started for the detection of aggregate-reactive antibodies to quantify the antibody levels within the serum. The calibration of the test is achieved with a test sample containing a known antibody, i.e. Rep-specific mouse-monoclonal antibody AB 8 (Bund et al., PNAS 2021 Vol. 118 No. 12 e2025830118).

FIGS. 2A-2C: Quantification of the antibody concentration against oligomeric Rep (oligomeric assay)
(a) Subgrouping for individuals (male and female) younger or equal to an age of 53 years at sample collection with 26 healthy control serum samples (HC) and 70 MS serum samples (MS); duplicate test per serum, 10 μl serum volume per single test. The measured antibody concentrations in MS patients is significantly increased when compared to healthy controls (tested by Mann Whitney U test, scatter plot with representation of median including 95% CI).
(b) Receiver Operator Curve (ROC) including ROC area (under curve), Specificity and Sensitivity (in %) and P-value for the ROC analysis
(c) Subtyping to the clinical courses of MS

FIGS. 3A-3B: Quantification of the antibody concentration against aggregate Rep (aggregate assay)
(a) Subgrouping for females younger or equal to an age of 40 years at sample collection with 11 healthy control serum samples (HC) and 27 MS serum samples (MS); duplicate test per serum, 10 μl serum volume per single test. The measured antibody concentrations in MS patients are significantly increased when compared to healthy controls (tested by Mann Whitney U test, scatter plot with representation of median including 95% CI).
(b) Receiver operator curve (ROC) including ROC area (under curve), Specificity and Sensitivity (in %) and P-value for the ROC analysis.

FIG. 4: Incidence rate and sex ratio of multiple sclerosis in Scotland 2010-2017
(reproduced from Kearns et al., J. of Neurology, 266, pp. 2376-2386 (2019))

FIG. 5: Epidemiology of Multiple Sclerosis in Austria
(reproduced from Salhofer-Polanyi et. Al., Neuroepidemiology, 49, pp. 40-44 (2017))

FIG. 6: Clinical Course of MS
(reproduced from Filippi, M., et al. Nat Rev Dis Primers 4, 43 (2018))

The National Multiple Sclerosis Society Advisory Committee on Clinical Trials in Multiple Sclerosis (MS) defined four clinical courses of MS: relapsing-remitting MS (RRMS), secondary progressive MS (SPMS), primary progressive MS (PPMS) and progressive relapsing MS (PRMS). A revision of these phenotypes has been proposed and includes clinically isolated syndrome (CIS) to denote those patients whose first clinical presentation has characteristics of inflammatory demyelination that could be MS but who do not fulfil its diagnostic criteria (pre-symptomatic phase). Within each subtype, disease can be classified as active or not active, which are defined by the occurrence of relapses or lesions detected using MRI.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1: Purification of the Non-Denatured H1MSB.1 Rep Protein Antigen

A nucleotide acid molecule encoding the full-length Rep open reading frame (ORF) identified within the H1MSB.1 genome (isolated from the brain of a Multiple Sclerosis patient, WT) or a mutant form thereof (mut) is each cloned into an expression plasmid (pEXP5-CT, Invitrogen, C-terminal 6×His-tag) enabling protein expression in E. coli (SoluBL21, Genlantis) upon IPTG induction (0.66 mM IPTG) for 18 h at 16° C. (LB medium, Ampicillin selection). The gene-expressing part was selected to either express the wild-type Rep amino acid sequence found in the H1MSB.1 genome to later on allow specific production of HRP-coupled, oligomeric test antigens or a variant with two point mutations (L27E and C154E) to generate adequate amounts of Biotin-coupled oligomeric test antigens. After induction of gene expression, the Rep-expressing cells are washed in 1×PBS pH 7.4 aliquoted, pelleted, and stored at −80° C. for further use.

For the separate purifications of the two Rep variants (WT and mut) based on IMAC/His affinity purification, the cells are lysed in lysis buffer (30 ml 1×PBS pH 7.4, 300 mM NaCl, 1% Triton X-100, 5 mM imidazole, 5 mM β-mercaptoethanol, containing Benzonase and proteinase inhibitors) by sonication on ice. After incubation for 30 min at 4° C. and centrifugation, the cleared lysate is loaded on an IMAC column (His60 Ni Superflow Resin, Clontech) and washed with 50 ml washing buffer (lysis buffer containing 35 mM imidazole, all purification steps performed at 4° C.). The wt Rep protein is eluted in 5 ml PBS lysis buffer pH 5 containing 300 mM imidazole while the Rep double mutant is eluted in 5 ml Carbonate buffer pH 9.5 containing 300 mM imidazole.

Quality of purification is determined by Coomassie protein staining and Western Blotting with anti-His and anti-Rep protein antibodies. The Rep-containing protein elutions are pooled to meet a final concentration of ˜1.2 mg/ml. The purity of the purified Rep target protein is densitometrically calculated and greater 98%.

### Example 2: Biotin-/HRP-Conjugation of the Rep for Bridging ELISA Serum Test

The purified wt and mutant Rep protein solutions consist of a mixture of an excess of Rep monomers, dimers and trimers (referred to as oligomers) and to a smaller extent of assemblies with ≥5 Rep sub-units (referred to as aggregates). The wt protein purification (pH 5) is re-buffered to pH 9.5 and subsequently subjected to biotinylation or HRP coupling. The preparation with the mutant Rep (pH 9.5) is subjected to conjugation to HPR without buffer exchange.

Coupling of biotin (N-Hydroxysuccinimide Ester (NHS)-biotin) is performed via primary amines of lysine residues at pH 9.5 with an excess of at least 20× when compared to the antigen (see e.g. documentation on Thermo Scientific™ EZ-Link™ NHS-Biotin biotinylation or Diner, I. et al., J Biol Chem. 2014-12-19; 289 (51): 35296-35313). HRP-coupling is performed based on a 2-step process, first adding N-succinimidyl S-acetylthioacetate (SATA) to the target antigens at primary amines of e.g. lysins to introduce protected sulfhydryl groups at pH 9.5. In a second step, after re-buffering by gelfiltration to a neutral pH (PBS at pH 7.5), maleimide-activated HRP is coupled to the antigen via the previously introduced sulfhydryl groups.

After conjugation, both the generated Biotin- and HRP-conjugated Rep antigen preparations containing oligomers and aggregates are individually subjected to size exclusion chromatography (SEC) based on a 30 cm Sephadex column to analyze the produced antigen preparations, to remove access Biotin and HRP label, and to re-buffer the antigens for long- and intermediate storage and for the final serum test in assay buffer. The elution of the conjugated Rep protein during SEC is monitored by UV/vis spectroscopy allowing size separation and for collection of fractions with oligomeric and aggregated Biotin- and HRP-conjugate preparations.

After SEC separation the HRP-conjugated Rep antigen preparation portions are used immediately or stored in PBS (di-Natriumhydrogenphosphate 8.1 mM, Potassiumdihydrogenphosphate 1.5 mM, Natriumchloride 136 mM, Potassiumchloride 2.7 mM, pH 7.3) plus same amount of glycerine at −20° C.

After SEC separation the biotin-conjugated Rep antigen preparation portions are used immediately or stored in carbonate buffer (Natriumhydrogencarbonate 0.1 M, Natriumchloride 0.5 M, pH 9.4) plus same amount of glycerine at −20° C.

To allow detection of antibodies reactive against oligomers, Biotin- as well as HRP-conjugated Rep oligomers are utilized together in an oligomeric bridging ELISA test setup (Oligomer Test). Detection of antibodies reactive against aggregates is performed by combining Biotin- as well as HRP-conjugated Rep aggregates in an aggregate bridging ELISA test setup (Aggregate Test).

### Example 3: Quantification of Anti-Rep Antibodies in Human Sera by Bridging ELISA Assay

Biotin-Rep oligomers or aggregates are immobilized separately on streptavidin-coated assay plates by incubation for 60 min at 20-25° C. After washing away any unbound material, the serum sample (or calibration/control antibodies) is pipetted onto the plate followed be addition of the HRP-Rep oligomers. After co-incubation for 2 h at 20-25° C., the unbound material is washed away. After this washing step, 3,3′,5,5′-Tetramethylbenzidine (TMB) substrate is added to the well, which reacts with the HRP and color is formed (substrate reaction). After a 30 minute incubation, the reaction is stopped with HCl and the plate is read using a plate reader at 450 nm. The amount of color generated is directly proportional to the amount of antibodies reacting with the Rep antigen in the sample. The concentration of aggregate- and/or oligomer-reactive antibodies in the sample of interest is calculated based on a calibration curve with a standard antibody (anti-Rep, mouse monoclonal antibody AB 8), which is run in parallel on each oligomer or aggregate test plate.

A calibration curve can be established by plotting standard concentration on the x-axis (linear scale) against the absorbance of the standards on the y-axis (linear scale). The antibody concentrations from patient samples can then be read off the calibration curve. A 4-parameter (4-PL, 4 Parameter Logistic) curve fit or a comparable method has been used for automatic data reduction. Standards and samples have been measured in duplicate or triplicate. If samples were pre-diluted different to the Standards, the concentration will be obtained by multiplying the value read off the calibration curve by the difference of the dilution factor.

The results are shown in FIG. 2 (oligomeric assay) and FIG. 3 (aggregate assay). Both tests show that the method of the present invention is suitable to provide a reliable distinction between MS and healthy controls.

### Example 4: Protocol for Stabilization of Biotin-Rep on Polystreptavidin-Coated Wells for Long-Term Storage of Test Plates and Reactivation

In an alternative test setup, the appropriate amount of Biotin Rep oligomers is immobilized on polystreptavidin-coated microtiter plate wells in 100 μl assay buffer (1-16 h, 21° C., on a shaking device), followed by 4 washing steps and stabilizing in stabilizing solution (200 μl per well; 20% saccharose, 0.06% 5-Bromo-5-nitro-1,3-dioxane, and optionally 1% (porcine) gelatine) for 1 h at 21° C. After stabilizing the Rep antigen on the microtiter plates, the solution is removed and the microtiter plates are dried at room temperature for at least 48 h and sealed in flat bags with desiccant for long-term storage at 2-8° C. (until reactivation with assay buffer for diagnostic use).

In the following Table 1 a comparison of the detected optical density (OD) of the oligomeric Rep assay with and without stabilization of the biotinylated Rep oligomer prior to sample testing. Biotinylated Rep oligomers were either immobilized on streptavidin-coated microtiter plates followed by stabilization, drying of the plates, storage for 7 days at 21° C. and reactivation for testing (“Stabilized”) or immobilized onto a microtiter plate just prior to testing following the standard protocol (“Reference”). Comparable OD values were observed for the standard antibody AB8 (calibration curve) and reference antibodies AB3 and AB11 as well for a ctrl. serum with or without addition of 50 ng/ml AB3.

TABLE 1

| Std. (ng/ml) | Stabilized | Reference |
|---|---|---|
| 200 | 2.673 | 2.615 |
| 100 | 1.337 | 1.427 |
| 50 | 0.712 | 0.777 |
| 25 | 0.417 | 0.465 |
| 12.5 | 0.278 | 0.292 |
| 6.25 | 0.188 | 0.219 |
| 3.13 | 0.158 | 0.159 |
| 0 | 0.102 | 0.134 |
| AB3 | 0.901 | 1.145 |
| AB3 | 0.84 | 1.113 |
| AB11 | 0.199 | 0.353 |
| AB11 | 0.179 | 0.324 |
| ctrl. serum | 0.091 | 0.126 |
| ctrl. serum | 0.087 | 0.112 |
| ctrl. serum + AB3 | 0.538 | 0.680 |
| ctrl. serum + AB3 | 0.572 | 0.685 |

In the following preferred embodiments of the present invention are described:
1. An immunoassay for the detection and/or measuring of an antibody against MSBI Rep protein in a subject's sample, wherein the method comprises:
(a) coating a solid support with a capture antigen being mutant MSBI Rep oligomer via a specific binding pair;
(b) contacting the capture antigen with the sample under conditions to form a capture antigen: anti-MSBI Rep antibody complex;
(c) removing unbound sample;
(d) contacting the capture antigen: anti-MSBI Rep antibody complex with a tracer compound being wild-type MSBI Rep oligomer conjugated to a detectable label or an anti-human antibody conjugated to a detectable label to form a capture antigen: anti-MSBI Rep antibody: tracer compound complex;
(e) removing unbound tracer compound;
(f) adding a dye, chemiluminescent or fluorescent substrate
(g) detecting and/or measuring the capture antigen: anti-MSBI Rep antibody: tracer compound complex via a detectable reaction of the substrate.
2. An immunoassay for the detection and/or measuring of an antibody against MSBI Rep protein in a subject's sample, wherein the method comprises:
(a) coating a solid support with a capture antigen being wild-type MSBI Rep aggregate via a specific binding pair;
(b) contacting the capture antigen with the sample under conditions to form a capture antigen: anti-MSBI Rep antibody complex;
(c) removing unbound sample;
(d) contacting the capture antigen: anti-MSBI Rep antibody complex with a tracer compound being wild-type MSBI Rep aggregate conjugated to a detectable label or an anti-human antibody conjugated to a detectable label to form a capture antigen: anti-MSBI Rep antibody: tracer compound complex;
(e) removing unbound tracer compound;
(f) adding a dye, chemiluminescent or fluorescent substrate
(g) detecting and/or measuring the capture antigen: anti-MSBI Rep antibody: tracer compound complex via a detectable reaction of the substrate.
3) The immunoassay of embodiment 1 or 2, wherein the specific binding pair is streptavidin/biotin wherein the streptavidin is immobilized on the solid support and the biotin is conjugated to the capture antigen.
4) The immunoassay of any of embodiments 1-3, wherein the detectable label is horseradish peroxidase.
5) The immunoassay of any of embodiments 1-4, wherein the substrate is 3,3′,5,5′-Tetramethylbenzidine.
6) The immunoassay of any of embodiments 1-5, wherein the anti-human antibody is an anti-human IgG or IgM antibody.
7) A kit, comprising (a) MSBI Rep protein or fragment thereof as a capture antigen that is immobilized via a specific binding pair to a solid phase, (b) MSBI Rep protein or fragment thereof that is conjugated to a detectable label or an anti-human antibody that is conjugated to a detectable label as a tracer compound, (c) a dye, chemiluminescent or fluorescent substrate and (d) instructions for using the kit in a method for diagnosing or monitoring Multiple Sclerosis.
8) The kit of embodiment 7, wherein the specific binding pair is streptavidin/biotin wherein the streptavidin is immobilized on the solid support and the biotin is conjugated to the capture antigen.
9) The kit of embodiment 7 or 8, wherein the detectable label is horseradish peroxidase.
10) The kit of any of embodiments 7-9, wherein the substrate is 3,3′,5,5′-Tetramethylbenzidine.
11) The kit of any of embodiments 7-10, wherein the anti-human antibody is an anti-human IgG or IgM antibody.
12) Use of the immunoassay of any of embodiments 1-6 or the kit of any of embodiments 7-10 for diagnosing or monitoring Multiple Sclerosis.

SEQUENCE SUMMARY

| SEQ | |
|---|---|
| ID | |
| NO | SEQUENCE |
| 1 | Amino acid sequence of Rep protein encoded by MSBI1.176 (wild-type) |
| | MSDLIVKDNALMNASYNLALVEQRLILLAIIEARETGKGINANDPLTVHASSYINQFNVERHTAY |
| | QALKDACKDLFARQFSYQEKRERGRINITSRWVSQIGYMDDTATVEIIFAPAVVPLITRLEEQFT |
| | QYDIEQISGLSSAYAVRMYELLICWRSTGKTPIIELDEFRKRIGVLDTEYTRTDNLKMRVIELALK |
| | QINEHTDITASYEQHKKGRVITGFSFKFKHKKQNSDKTPKNSDSSPRIVKHSQIPTNIVKQPEN |
| | AKMSDLEHRASRVTGEIMRNRLSDRFKQGDESAIDMMKRIQSEIITDAIADQWESKLEEFGVVF |
| 2 | Nucleotide sequence of mutant Rep protein encoding MSBI1 Rep |
| | 27/154E (mutant DNA) |
| | ATGAGCGACCTGATCGTGAAAGACAATGCCCTGATGAACGCCTCCTACAACCTGGCACTG |
| | GTCGAACAGAGACTGATTGAGCTGGCTATCATCGAGGCAAGGGAGACCGGCAAGGGCAT |
| | CAACGCCAATGACCCCCTGACAGTGCACGCCAGCTCCTACATCAACCAGTTTAATGTGGA |
| | GCGCCACACCGCCTATCAGGCCCTGAAGGACGCCTGCAAGGATCTGTTTGCCCGGCAGT |
| | TCAGCTACCAGGAGAAGCGGGAGAGAGGCAGGATCAACATCACAAGCAGATGGGTGTCC |
| | CAGATCGGCTATATGGACGATACCGCCACAGTGGAGATCATCTTTGCACCAGCAGTGGTG |
| | CCTCTGATCACCAGGCTGGAGGAGCAGTTCACACAGTACGACATCGAGCAGATCTCCGG |
| | ACTGTCTAGCGCCTACGCCGTGCGCATGTATGAGCTGCTGATCGAGTGGCGGTCTACCG |
| | GCAAGACACCTATCATCGAGCTGGATGAGTTCCGCAAGCGGATCGGCGTGCTGGACACC |
| | GAGTACACCAGAACAGATAACCTGAAGATGAGAGTGATCGAGCTGGCCCTGAAGCAGAT |
| | CAATGAGCACACCGATATCACAGCCTCTTATGAGCAGCACAAGAAGGGCCGCGTGATCA |
| | CCGGCTTCAGCTTTAAGTTCAAGCACAAGAAGCAGAACTCTGACAAGACACCAAAGAATA |
| | GCGATTCCTCTCCCCGGATCGTGAAGCACAGCCAGATCCCTACCAACATCGTGAAGCAG |
| | CCAGAGAATGCCAAGATGTCCGACCTGGAGCACAGGGCATCTAGGGTGACAGGCGAGAT |
| | CATGAGAAATAGGCTGAGCGATCGGTTCAAGCAGGGCGACGAGTCCGCCATCGATATGA |
| | TGAAGAGAATCCAGTCCGAGATCATCACCGACGCCATCGCCGATCAGTGGGAATCTAAAC |
| | TGGAAGAGTTTGGAGTCGTGTTTGGAGCACATCACCATCATCATCACTGA |
| 3 | Amino acid sequence of Rep protein encoded by MSBI1 Rep 27/154E |
| | (mutant protein) |
| | MSDLIVKDNALMNASYNLALVEQRLIELAIIEARETGKGINANDPLTVHASSYINQFNVERHTAY |
| | QALKDACKDLFARQFSYQEKRERGRINITSRWVSQIGYMDDTATVEIIFAPAVVPLITRLEEQFT |
| | QYDIEQISGLSSAYAVRMYELLIEWRSTGKTPIIELDEFRKRIGVLDTEYTRTDNLKMRVIELALK |
| | QINEHTDITASYEQHKKGRVITGFSFKFKHKKQNSDKTPKNSDSSPRIVKHSQIPTNIVKQPEN |
| | AKMSDLEHRASRVTGEIMRNRLSDRFKQGDESAIDMMKRIQSEIITDAIADQWESKLEEFGVVF |
| 4 | Amino acid sequence of Rep protein encoded by MSBI2.176 (wild-type) |
| | MSKLVVKDNALMNASYNLDLVEQRLILLAIIEARESGKGINANDPLTVHAESYINQFGVHRVTAYQALKDA |
| | CDNLFARQFSYQSKSEKGNIQNHRSRWVSEIIYIDTEATVKIIFAPAIVPLITRLEEQFTKYDIEQISDLSSAY |
| | AIRLYELLICWRSTGKTPIIGLGEFRNRVGVLDSEYHRIAHLKERVIEHSIKQINEHTDITATYEQHKKGRTIT |
| | GFSFKFKQKKPKQAEIATETPKTATNDPDTTKPLTEPQIAKYSMILCKLGSISDLSNFPDYPAFANWIGNIL |
| | RNPEKADEQIAKRIFTALKTETDYSKKN |
| 5 | Amino acid sequence of Rep protein encoded by MSBI2.176 Rep |
| | 27/155E (mutant protein) |
| | MSKLVVKDNALMNASYNLDLVEQRLIELAIIEARESGKGINANDPLTVHAESYINQFGVHRVTAYQALKDA |
| | CDNLFARQFSYQSKSEKGNIQNHRSRWVSEIIYIDTEATVKIIFAPAIVPLITRLEEQFTKYDIEQISDLSSAY |
| | AIRLYELLIEWRSTGKTPIIGLGEFRNRVGVLDSEYHRIAHLKERVIEHSIKQINEHTDITATYEQHKKGRTIT |
| | GFSFKFKQKKPKQAEIATETPKTATNDPDTTKPLTEPQIAKYSMILCKLGSISDLSNFPDYPAFANWIGNIL |
| | RNPEKADEQIAKRIFTALKTETDYSKKN |
| 6 | Nucleotide sequence of Rep protein MSBI 1.176 (wild-type): |
| | ATGAGCGATTTAATAGTAAAAGATAACGCCCTAATGAATGCTAGTTATAA |
| | CTTAGCTTTGGTTGAACAGAGGTTAATTCTATTAGCAATCATAGAAGCGA |
| | GAGAAACAGGCAAAGGGATTAATGCCAATGATCCTTTAACAGTTCATGCA |
| | AGTAGCTATATCAATCAATTTAACGTAGAAAGGCATACGGCATATCAAGC |
| | CCTCAAAGATGCTTGTAAAGACTTGTTTGCCCGTCAATTCAGTTACCAAG |
| | AAAAGCGAGAACGAGGACGAATTAATATTACAAGTCGATGGGTTTCGCAA |
| | ATTGGCTATATGGACGATACAGCAACCGTTGAGATTATTTTTGCCCCTGC |
| | GGTTGTTCCTCTGATTACACGGCTAGAGGAACAGTTCACCCAGTACGATA |
| | TTGAGCAAATTAGCGGTTTATCGAGTGCATATGCTGTTCGTATGTACGAA |
| | CTGCTGATTTGTTGGCGTAGCACAGGCAAAACACCAATTATTGAGCTAGA |
| | CGAGTTTAGAAAGCGAATAGGTGTTTTAGATACTGAATACACTAGAACAG |
| | ATAATTTAAAGATGCGAGTTATTGAATTAGCCCTAAAACAAATCAACGAA |
| | CATACAGACATCACAGCAAGCTATGAACAACACAAAAAAGGGCGAGTGAT |
| | TACAGGATTCTCATTCAAGTTTAAGCACAAGAAACAAAACAGCGATAAAA |
| | CGCCAAAAAATAGCGATTCTAGCCCACGTATCGTAAAACATAGTCAAATC |
| | CCTACCAACATTGTAAAACAGCCTGAAAACGCCAAAATGAGCGATTTAGA |
| | ACATAGAGCGAGCCGTGTTACAGGGGAAATAATGCGAAATCGTCTGTCAG |
| | ATCGGTTTAAACAAGGCGATGAATCAGCAATCGACATGATGAAACGTATT |
| | CAAAGTGAAATAATAACCGATGCAATAGCAGACCAGTGGGAAAGCAAACT |
| | GGAGGAGTTTGGCGTGGTTTTTTAG |
| 7 | Nucleotide sequence of MSBI1.176 Rep (wild-type) codon-optimized for |
| | expression in E. coli: |
| | ATGAGCGACCTGATCGTGAAAGACAATGCCCTGATGAACGCCTCCTACAACCTGGCACTGGTCGAACAGAGACTG |
| | ATTCTGCTGGCTATCATCGAGGCAAGGGAGACCGGCAAGGGCATCAACGCCAATGACCCCCTGACAGTGCACGCC |
| | AGCTCCTACATCAACCAGTTTAATGTGGAGCGCCACACCGCCTATCAGGCCCTGAAGGACGCCTGCAAGGATCTG |
| | TTTGCCCGGCAGTTCAGCTACCAGGAGAAGCGGGAGAGAGGCAGGATCAACATCACAAGCAGATGGGTGTCCCAG |
| | ATCGGCTATATGGACGATACCGCCACAGTGGAGATCATCTTTGCACCAGCAGTGGTGCCTCTGATCACCAGGCTG |
| | GAGGAGCAGTTCACACAGTACGACATCGAGCAGATCTCCGGACTGTCTAGCGCCTACGCCGTGCGCATGTATGAG |
| | CTGCTGATCTGTTGGCGGTCTACCGGCAAGACACCTATCATCGAGCTGGATGAGTTCCGCAAGCGGATCGGCGTG |
| | CTGGACACCGAGTACACCAGAACAGATAACCTGAAGATGAGAGTGATCGAGCTGGCCCTGAAGCAGATCAATGAG |
| | CACACCGATATCACAGCCTCTTATGAGCAGCACAAGAAGGGCCGCGTGATCACCGGCTTCAGCTTTAAGTTCAAG |
| | CACAAGAAGCAGAACTCTGACAAGACACCAAAGAATAGCGATTCCTCTCCCCGGATCGTGAAGCACAGCCAGATC |
| | CCTACCAACATCGTGAAGCAGCCAGAGAATGCCAAGATGTCCGACCTGGAGCACAGGGCATCTAGGGTGACAGGC |
| | GAGATCATGAGAAATAGGCTGAGCGATCGGTTCAAGCAGGGCGACGAGTCCGCCATCGATATGATGAAGAGAATC |
| | CAGTCCGAGATCATCACCGACGCCATCGCCGATCAGTGGGAATCTAAACTGGAAGAGTTTGGAGTCGTGTTT |
| | GGA GCA CATCACCATCATCATCAC TGA |
| | GGA GCA = Glycine-Alanine-Linker |
| 8 | Nucleotide Sequence of MSBI1.176 Rep (mutant) codon-optimized for |
| | expression in E. coli: |
| | ATGAGCGACCTGATCGTGAAAGACAATGCCCTGATGAACGCCTCCTACAACCTGGCACTGGTCGAACAGAGACTG |
| | ATTgagCTGGCTATCATCGAGGCAAGGGAGACCGGCAAGGGCATCAACGCCAATGACCCCCTGACAGTGCACGCC |
| | AGCTCCTACATCAACCAGTTTAATGTGGAGCGCCACACCGCCTATCAGGCCCTGAAGGACGCCTGCAAGGATCTG |
| | TTTGCCCGGCAGTTCAGCTACCAGGAGAAGCGGGAGAGAGGCAGGATCAACATCACAAGCAGATGGGTGTCCCAG |
| | ATCGGCTATATGGACGATACCGCCACAGTGGAGATCATCTTTGCACCAGCAGTGGTGCCTCTGATCACCAGGCTG |
| | GAGGAGCAGTTCACACAGTACGACATCGAGCAGATCTCCGGACTGTCTAGCGCCTACGCCGTGCGCATGTATGAG |
| | CTGCTGATCgagTGGCGGTCTACCGGCAAGACACCTATCATCGAGCTGGATGAGTTCCGCAAGCGGATCGGCGTG |
| | CTGGACACCGAGTACACCAGAACAGATAACCTGAAGATGAGAGTGATCGAGCTGGCCCTGAAGCAGATCAATGAG |
| | CACACCGATATCACAGCCTCTTATGAGCAGCACAAGAAGGGCCGCGTGATCACCGGCTTCAGCTTTAAGTTCAAG |
| | CACAAGAAGCAGAACTCTGACAAGACACCAAAGAATAGCGATTCCTCTCCCCGGATCGTGAAGCACAGCCAGATC |
| | CCTACCAACATCGTGAAGCAGCCAGAGAATGCCAAGATGTCCGACCTGGAGCACAGGGCATCTAGGGTGACAGGC |
| | GAGATCATGAGAAATAGGCTGAGCGATCGGTTCAAGCAGGGCGACGAGTCCGCCATCGATATGATGAAGAGAATC |
| | CAGTCCGAGATCATCACCGACGCCATCGCCGATCAGTGGGAATCTAAACTGGAAGAGTTTGGAGTCGTGTTT |
| | GGA GCA CATCACCATCATCATCAC TGA |
| | GGA GCA = Glycine-Alanine-Linker |
| | CATCACCATCATCATCAC = 6xHis tag |

1. An immunoassay for the detection and/or measuring of an antibody against MSBI Rep protein in a subject's sample, wherein the method comprises:
(a) coating a solid support with a capture antigen being mutant MSBI Rep oligomer via a specific binding pair;
(b) contacting the capture antigen with the sample under conditions to form a capture antigen: anti-MSBI Rep antibody complex;
(c) removing unbound sample;
(d) contacting the capture antigen: anti-MSBI Rep antibody complex with a tracer compound being wild-type MSBI Rep oligomer conjugated to a detectable label or an anti-human antibody conjugated to a detectable label to form a capture antigen: anti-MSBI Rep antibody: tracer compound complex;
(e) removing unbound tracer compound;
(f) adding a dye, chemiluminescent or fluorescent substrate; and
(g) detecting and/or measuring the capture antigen: anti-MSBI Rep antibody: tracer compound complex via a detectable reaction of the substrate.

2. The immunoassay of claim 1, wherein the mutant MSBI Rep oligomer contains 1-4 Rep subunits and the wild-type MSBI Rep oligomer contains 5-9 Rep subunits.

3. An immunoassay for the detection and/or measuring of an antibody against MSBI Rep protein in a subject's sample, wherein the method comprises:
(a) coating a solid support with a capture antigen being wild-type MSBI Rep aggregate via a specific binding pair;
(b) contacting the capture antigen with the sample under conditions to form a capture antigen: anti-MSBI Rep antibody complex;
(c) removing unbound sample;
(d) contacting the capture antigen: anti-MSBI Rep antibody complex with a tracer compound being wild-type MSBI Rep aggregate conjugated to a detectable label or an anti-human antibody conjugated to a detectable label to form a capture antigen: anti-MSBI Rep antibody: tracer compound complex;
(e) removing unbound tracer compound;
(f) adding a dye, chemiluminescent or fluorescent substrate
(g) detecting and/or measuring the capture antigen: anti-MSBI Rep antibody: tracer compound complex via a detectable reaction of the substrate.

4. The immunoassay of claim 3, wherein the wild-type MSBI Rep aggregate contains 5-9 Rep subunits.

5. The immunoassay of claim 1, wherein the specific binding pair is streptavidin/biotin wherein the streptavidin is immobilized on the solid support and the biotin is conjugated to the capture antigen.

6. The immunoassay of claim 1, wherein the detectable label is horseradish peroxidase.

7. The immunoassay of claim 1, wherein the substrate is 3,3′,5,5′-Tetramethylbenzidine.

8. The immunoassay of claim 1, wherein the anti-human antibody is an anti-human IgG or IgM antibody.

9. A kit, comprising (a) MSBI Rep protein or fragment thereof as a capture antigen that is immobilized via a specific binding pair to a solid phase, (b) MSBI Rep protein or fragment thereof that is conjugated to a detectable label or an anti-human antibody that is conjugated to a detectable label as a tracer compound, (c) a dye, chemiluminescent or fluorescent substrate and (d) instructions for using the kit in a method for diagnosing or monitoring Multiple Sclerosis.

10. The kit of claim 9, wherein the specific binding pair is streptavidin/biotin wherein the streptavidin is immobilized on the solid support and the biotin is conjugated to the capture antigen.

11. The kit of claim 9, wherein the detectable label is horseradish peroxidase.

12. The kit of claim 9, wherein the substrate is 3,3′,5,5′-Tetramethylbenzidine.

13. The kit of claim 9, wherein the anti-human antibody is an anti-human IgG or IgM antibody.

14. Use of the kit of claim 9, for diagnosing or monitoring Multiple Sclerosis.

15. Use of the immunoassay of claim 1, for diagnosing or monitoring Multiple Sclerosis.


SEQUENCE LISTING (8 sequences)

SEQ ID NO: 1 - protein, 324 aa, Homo sapiens
MSDLIVKDNALMNASYNLALVEQRLILLAIIEARETGKGINANDPLTVHASSYINQFNVERHTAYQALKDACKDLFARQFSYQEKRERGRINITSRWVSQIGYMDDTATVEIIFAPAVVPLITRLEEQFTQYDIEQISGLSSAYAVRMYELLICWRSTGKTPIIELDEFRKRIGVLDTEYTRTDNLKMRVIELALKQINEHTDITASYEQHKKGRVITGFSFKFKHKKQNSDKTPKNSDSSPRIVKHSQIPTNIVKQPENAKMSDLEHRASRVTGEIMRNRLSDRFKQGDESAIDMMKRIQSEIITDAIADQWESKLEEFGVVF

SEQ ID NO: 2 - DNA, 999 bp, synthetic construct
  misc_feature 1..999: note = Rep protein mutant
  source 1..999: mol_type = other DNA
atgagcgacctgatcgtgaaagacaatgccctgatgaacgcctcctacaacctggcactggtcgaacagagactgattgagctggctatcatcgaggcaagggagaccggcaagggcatcaacgccaatgaccccctgacagtgcacgccagctcctacatcaaccagtttaatgtggagcgccacaccgcctatcaggccctgaaggacgcctgcaaggatctgtttgcccggcagttcagctaccaggagaagcgggagagaggcaggatcaacatcacaagcagatgggtgtcccagatcggctatatggacgataccgccacagtggagatcatctttgcaccagcagtggtgcctctgatcaccaggctggaggagcagttcacacagtacgacatcgagcagatctccggactgtctagcgcctacgccgtgcgcatgtatgagctgctgatcgagtggcggtctaccggcaagacacctatcatcgagctggatgagttccgcaagcggatcggcgtgctggacaccgagtacaccagaacagataacctgaagatgagagtgatcgagctggccctgaagcagatcaatgagcacaccgatatcacagcctcttatgagcagcacaagaagggccgcgtgatcaccggcttcagctttaagttcaagcacaagaagcagaactctgacaagacaccaaagaatagcgattcctctccccggatcgtgaagcacagccagatccctaccaacatcgtgaagcagccagagaatgccaagatgtccgacctggagcacagggcatctagggtgacaggcgagatcatgagaaataggctgagcgatcggttcaagcagggcgacgagtccgccatcgatatgatgaagagaatccagtccgagatcatcaccgacgccatcgccgatcagtgggaatctaaactggaagagtttggagtcgtgtttggagcacatcaccatcatcatcactga

SEQ ID NO: 3 - protein, 324 aa, synthetic construct
  REGION 1..324: note = MSBI1 Rep 27/154E mutant
MSDLIVKDNALMNASYNLALVEQRLIELAIIEARETGKGINANDPLTVHASSYINQFNVERHTAYQALKDACKDLFARQFSYQEKRERGRINITSRWVSQIGYMDDTATVEIIFAPAVVPLITRLEEQFTQYDIEQISGLSSAYAVRMYELLIEWRSTGKTPIIELDEFRKRIGVLDTEYTRTDNLKMRVIELALKQINEHTDITASYEQHKKGRVITGFSFKFKHKKQNSDKTPKNSDSSPRIVKHSQIPTNIVKQPENAKMSDLEHRASRVTGEIMRNRLSDRFKQGDESAIDMMKRIQSEIITDAIADQWESKLEEFGVVF

SEQ ID NO: 4 - protein, 319 aa, Homo sapiens
MSKLVVKDNALMNASYNLDLVEQRLILLAIIEARESGKGINANDPLTVHAESYINQFGVHRVTAYQALKDACDNLFARQFSYQSKSEKGNIQNHRSRWVSEIIYIDTEATVKIIFAPAIVPLITRLEEQFTKYDIEQISDLSSAYAIRLYELLICWRSTGKTPIIGLGEFRNRVGVLDSEYHRIAHLKERVIEHSIKQINEHTDITATYEQHKKGRTITGFSFKFKQKKPKQAEIATETPKTATNDPDTTKPLTEPQIAKYSMILCKLGSISDLSNFPDYPAFANWIGNILRNPEKADEQIAKRIFTALKTETDYSKKN

SEQ ID NO: 5 - protein, 319 aa, synthetic construct
  REGION 1..319: note = MSBI2.176 Rep 27/155E mutant
MSKLVVKDNALMNASYNLDLVEQRLIELAIIEARESGKGINANDPLTVHAESYINQFGVHRVTAYQALKDACDNLFARQFSYQSKSEKGNIQNHRSRWVSEIIYIDTEATVKIIFAPAIVPLITRLEEQFTKYDIEQISDLSSAYAIRLYELLIEWRSTGKTPIIGLGEFRNRVGVLDSEYHRIAHLKERVIEHSIKQINEHTDITATYEQHKKGRTITGFSFKFKQKKPKQAEIATETPKTATNDPDTTKPLTEPQIAKYSMILCKLGSISDLSNFPDYPAFANWIGNILRNPEKADEQIAKRIFTALKTETDYSKKN

SEQ ID NO: 6 - DNA, 975 bp, Homo sapiens
  source 1..975: mol_type = genomic DNA
atgagcgatttaatagtaaaagataacgccctaatgaatgctagttataacttagctttggttgaacagaggttaattctattagcaatcatagaagcgagagaaacaggcaaagggattaatgccaatgatcctttaacagttcatgcaagtagctatatcaatcaatttaacgtagaaaggcatacggcatatcaagccctcaaagatgcttgtaaagacttgtttgcccgtcaattcagttaccaagaaaagcgagaacgaggacgaattaatattacaagtcgatgggtttcgcaaattggctatatggacgatacagcaaccgttgagattatttttgcccctgcggttgttcctctgattacacggctagaggaacagttcacccagtacgatattgagcaaattagcggtttatcgagtgcatatgctgttcgtatgtacgaactgctgatttgttggcgtagcacaggcaaaacaccaattattgagctagacgagtttagaaagcgaataggtgttttagatactgaatacactagaacagataatttaaagatgcgagttattgaattagccctaaaacaaatcaacgaacatacagacatcacagcaagctatgaacaacacaaaaaagggcgagtgattacaggattctcattcaagtttaagcacaagaaacaaaacagcgataaaacgccaaaaaatagcgattctagcccacgtatcgtaaaacatagtcaaatccctaccaacattgtaaaacagcctgaaaacgccaaaatgagcgatttagaacatagagcgagccgtgttacaggggaaataatgcgaaatcgtctgtcagatcggtttaaacaaggcgatgaatcagcaatcgacatgatgaaacgtattcaaagtgaaataataaccgatgcaatagcagaccagtgggaaagcaaactggaggagtttggcgtggttttttag

SEQ ID NO: 7 - DNA, 999 bp, synthetic construct
  misc_feature 1..999: note = MSBI1.176 Rep codon optimized
  source 1..999: mol_type = other DNA
atgagcgacctgatcgtgaaagacaatgccctgatgaacgcctcctacaacctggcactggtcgaacagagactgattctgctggctatcatcgaggcaagggagaccggcaagggcatcaacgccaatgaccccctgacagtgcacgccagctcctacatcaaccagtttaatgtggagcgccacaccgcctatcaggccctgaaggacgcctgcaaggatctgtttgcccggcagttcagctaccaggagaagcgggagagaggcaggatcaacatcacaagcagatgggtgtcccagatcggctatatggacgataccgccacagtggagatcatctttgcaccagcagtggtgcctctgatcaccaggctggaggagcagttcacacagtacgacatcgagcagatctccggactgtctagcgcctacgccgtgcgcatgtatgagctgctgatctgttggcggtctaccggcaagacacctatcatcgagctggatgagttccgcaagcggatcggcgtgctggacaccgagtacaccagaacagataacctgaagatgagagtgatcgagctggccctgaagcagatcaatgagcacaccgatatcacagcctcttatgagcagcacaagaagggccgcgtgatcaccggcttcagctttaagttcaagcacaagaagcagaactctgacaagacaccaaagaatagcgattcctctccccggatcgtgaagcacagccagatccctaccaacatcgtgaagcagccagagaatgccaagatgtccgacctggagcacagggcatctagggtgacaggcgagatcatgagaaataggctgagcgatcggttcaagcagggcgacgagtccgccatcgatatgatgaagagaatccagtccgagatcatcaccgacgccatcgccgatcagtgggaatctaaactggaagagtttggagtcgtgtttggagcacatcaccatcatcatcactga

SEQ ID NO: 8 - DNA, 999 bp, synthetic construct; identical to SEQ ID NO: 2
  misc_feature 1..999: note = MSBI1.176 Rep mutant codon optimized
  source 1..999: mol_type = other DNA